(19) United States
(12) Patent Application Publication
(10) Pub. No.: US 2007/0218555 A1
(43) Pub. Date: Sep. 20, 2007

(54) Anti-Microbial Activity Of Biologically Stabilized Silver Nano Particles

(76) Inventor: Kishore Madhukar Paknikar, Pune (IN)

Correspondence Address: HEDMAN & COSTIGAN P.C., 1185 AVENUE OF THE AMERICAS, NEW YORK, NY, 10036, US

(21) Appl. No.: 11/547,886

(22) PCT Filed: May 12, 2005
(86) PCT No.: PCT/IN05/00155

§ 371 Date: Oct. 5, 2006

(30) Foreign Application Priority Data
May 12, 2004 (IN) 548/MUM/2004

(51) Int. Cl. C12N 5/00; C01G 5/00

(52) U.S. Cl. 435/410; 423/659; 435/317.1

(57) ABSTRACT

An antimicrobial formulation containing biologically stabilized silver nano particles stabilized by a ‘green’ biological route with an average size 1-100 nm in a carrier in which the concentration is 1 to 6 ppm.

This invention relates to an antimicrobial formulation.

What is envisaged in accordance with this invention is an antimicrobial formulation containing silver.

### BACKGROUND OF INVENTION

The usefulness of silver as an antimicrobial agent has been known for a long time. For thousands of years silver has been used as a healing and anti-bacterial agent by civilizations throughout the world. Its medical, preservative and restorative powers can be traced as far back as the ancient Greek and Roman Empires. Long before the development of modern pharmaceuticals, silver was employed as a germicide and antibiotic:
The Greeks used silver vessels to keep water and other liquids fresh. The writings Herodotus, the Greek philosopher and historian, date the use of silver to before the birth of Christ.
The Roman Empire stored wine in silver urns to prevent spoilage.
The use of silver is mentioned in ancient Indian and Egyptian writings.
In the Middle Ages, silverware protected the wealthy from the full brunt of the plague.
Before the advent of modern germicides and antibiotics, it was known that disease-causing pathogens could not survive in the presence of silver. Consequently, silver was used in dishware, drinking vessels and eating utensils.
In particular, the wealthy stored and ate their food from silver vessels to keep bacteria from growing.
The Chinese emperors and their courts ate with silver chopsticks.
The Druids have left evidence of their use of silver.
Settlers in the Australian outback suspend silverware in their water tanks to retard spoilage.
Pioneers trekking across the American West found that if they placed silver or copper coins in their casks of drinking water, it kept the water safe from bacteria, algae, etc.
All along the frontier, silver dollars were put in milk to keep it fresh.
Silver leaf was used to combat infection in wounds sustained by troops during World War 1.
Prior to the introduction of antibiotics, Colloidal silver was used widely in hospitals and has been known as a bactericide for at least 1200 years.
In the early 1800s, doctors used silver sutures in surgical wounds with very successful results.
In Ayurvedic medicine, silver is used in small amounts as a tonic, elixir or rejuvenating agent for patients debilitated by age or disease.

Not until the late 1800's did eastern scientists re-discover what had been known for thousands of years-that silver is a powerful germ fighter. Medicinal silver compounds were then developed and silver became commonly used as a medicine. By the early part of the 1900s, the use of silver as an antibacterial substance was becoming widespread. By 1940 there were approximately four dozen different silver compounds on the market being used to treat every known infectious disease. These were available in oral, injectable, and topical forms. However, these medicinal silver preparations caused a discoloration of the skin called argyria specially certain types of protein-bound silver compounds and improperly prepared and unstable compositions.

New knowledge of body chemistry gave rise to the enormous array of applications for colloidal disinfectants and medicines and for on-going research into the capabilities and possibilities for silver colloids. However, Silver's “new-found” fame as a superior infection-fighting agent was short lived.

During the 1930s, synthetically manufactured drugs began to make their appearance and the profits, together with the simplicities of manufacturing this new source of treatment, became a powerful force in the marketplace. There was much excitement over the new ‘wonder drugs’ and at that time, no antibiotic-resistant strains of disease organisms had surfaced. Silver quickly lost its status to modern antibiotics.

The use of some silver preparations in mainstream medicine survived. Among them are the use of dilute silver nitrate in newborn babies' eyes to protect from infection and the use of “Silvadine”, a silver based salve, in burn wards to kill infection. Silver based bandages have also been approved by the FDA and licensed for sale. Other uses that did not lose favor include:
Silver water purification filters and tablets are used to prevent growth of algae and bacteria.
Electrical ionization units that impregnate the water with silver and copper ions are used to sanitize pool water without the harsh effects of chlorine.
Silver has been used to sterilize recycled water on space vehicles.
The Swiss use silver filters in homes and offices.
Municipalities use silver in treatment of sewage.
Silver is a popular agent in the fight against airborne toxins as well other industrial poisons.

But for the most part, with the discovery of pharmaceutical antibiotics, interest in Silver as an anti-microbial agent declined almost to the point of extinction.

Silver re-emerged as an adjunct to antibiotic treatment as a result of the notable work of Dr. Margraf who found that the use of diluted silver nitrate to a 5 percent solution was found to kill invasive burn bacteria and permitted wounds to heal. Importantly, resistant strains did not appear. Silver nitrate was widely used in 1960s for the treatment of burn victims by Moyer. But, silver nitrate was far from ideal. Eventually, it was not considered as an ideal antimicrobial agent owing to many complications such as neutralization of Ag⁺ ions with Cl⁻, HCO₋₃ and protein anions in the body fluids (thus reducing its microbicidal activity), and development of a cosmetic abnormality, viz. argyria caused by precipitation of silver salts in the skin leading to blue gray coloration.

Silver sulphadiazine was developed (Silvadene, Marion Laboratories) which is now used in 70 percent of burn centers. Discovered by Dr. Charles Fox of Columbia University, sulphadiazine has also been successful in treating cholera, malaria and syphilis. It also stops the herpes virus, which is responsible for cold sores, shingles and worse.

Because of the research showing colloidal silver's superior performance in fighting microbes, it has attracted the attention of leading scientists and medical researchers throughout the world. Its benefits are now stirring new interest as 50 prominent doctors are currently researching the efficacy and applications of colloidal silver in human health. As a result, many interesting studies have emerged.

According to experts, no microorganism ever tested has been able to stay alive for more than six minutes when exposed directly to colloidal silver.

Science Digest cites colloidal silver as “ . . . a wonder of modern medicine”, and further states “Antibiotics kill perhaps a half dozen different disease organisms, but silver kills some 650. Resistant strains fail to develop. Moreover, silver is virtually non-toxic. Colloidal silver, used as an anti-microbial agent, will not create super bugs as antibiotics do.” Alfred Searle, founder of the giant Searle Pharmaceuticals (now Monsanto) stated, “Applying colloidal silver to human subjects has been done in a large number of cases with astonishingly successful results. For internal administration, it has the advantage being rapidly fatal to pathogens without toxic action on its host. It is quite stable.” Further information indicates that Colloidal Silver does not cause harmful interactions with other medications or topical treatments. In laboratory tests with colloidal silver, bacteria, viruses, and fungal organisms are killed within minutes of contact. Larry C. Ford, M. D. of the Department of Obstetrics and Genecology, UCLA School of Medicine, Centre For The Health Sciences reported in Nov. 1, 1988, “I tested them (the silver solutions) using standard anti-microbial tests for disinfectants. The silver solutions were anti-bacterial for concentrations of 105 organisms per ml of Streptococcus Pyogenes, Staphylococcus Aureus, Neisseria Gonorrhea, Gardnerella Vaginalis, Salmonella Typhi and other enteric pathogens, and fungicidal for Candida Albicans, Candida Globata and M. Furfur.”

Because of the many organisms that have developed strains resistant to modern antibiotics, Dr. Robert Becker's finding is of particular importance. Becker, of Syracuse University stated, “All of the organisms that we tested were sensitive to the electrically generated silver ions, including some that were resistant to all known antibiotics. In no case were any undesirable side effects of the silver treatment apparent.”

The potential of colloidal silver is significant because unlike antibiotics, which are specific only to bacteria, Colloidal Silver disables certain enzymes needed by anaerobic bacteria, viruses, yeasts, and fungus resulting in the destruction of these enzymes. Further indication is that these bacteria cannot develop a resistance to silver, as they do with antibiotics, because silver attacks their food source, rather than them directly.

However it has now been realized, that both silver-nitrate and silver-sulfadiazine impair fibroblast and epithelial proliferation, eventually arresting the healing process. Attempts at finding better remedies with silver have met with a limited success. Some interesting reports have appeared in the past few years that describe application of silver coated films in burns treatment. These films were prepared by vapor deposition technique so as to get a thickness of ca. 300 nm. In one of these studies the films purportedly contained chemically capped nano crystalline silver, with a typical grain size of ˜50 nm. Such films deliver a sustained dose of high (5000-10000 mg/l) concentrations of silver, which have cytotoxic effects. Significant absorption of silver ions through burn wound can occur when patients are treated with topical silver containing preparations. The estimated silver concentration in liver was 14 mg/gm when silver preparations with cream base containing up to 3000 μg Ag⁺/gm were used. The effect of silver nitrate on human dermal fibroblast cells were studied by Hidalgo et al and it was found that low concentration (8.2 μM/l) of silver ions shows inhibitory effect.

There is therefore a need of a silver containing preparation which can be used as an antimicrobial agent effectively without having any cytotoxic effects and without having any added substances which are not bio-compatible.

### SUMMARY OF THE INVENTION

It is the object of this invention to provide an antimicrobial formulation containing biologically stabilized silver nano particles stabilized by a ‘green’ biological route with an average size 1-100 nm in a carrier in which the concentration is 1 to 6 ppm.

It is another object of this invention to provide an antimicrobial formulation containing biologically stabilised silver nano particles which (a) exhibits antimicrobial activity at very low effective concentrations (owing to their extremely high surface area) and (b) is not cytotoxic, at these concentrations.

### STATEMENT OF THE INVENTION

According to this invention there is provided an antimicrobial formulation comprising
(1) biologically stabilized silver nano particles in the size range of 1 to 100 nm and
(2) a carrier in which the concentration of the said biologically stabilized silver nano particles is in the range of 1 to 6 ppm.

Typically, the silver nano particles are stabilized biologically with an aqueous solution of macerated plant tissue cells.

In accordance with one embodiment of the invention, the aqueous solution is diluted in deionised water up to ten folds.

Typically, the plant tissue is at least one plant tissue selected from a group of plant tissues which include leaves, roots, stems, flowers and fruits of the following plants Alfa alfa, Babul (Acacia arabica), Coriander (Coriandrum sativum), East Indian Rosebay (Ervatamia coronaria), Hog weed (Boerhavia diffusa), Indian Barberry (Berberris aristata), Marigold (Calendula officinalis), Parsley (Petroselinum sativum), Rough Chaff (Achyranthes aspera), Tenner's Cassia (Cassia auriculata), Lavender, Bahera (Terminalia belerica), Fennel (Fenniculum vulgare), Horsetail (Equisetum arvense), Raspberry (Rubus idaeus), Aloe vera (Aloe barbadensis), Golden seal (Hydrastis canadensis), Garlic (Allium sativum), Echinacea spp., Eyebright (Euphrasis officinalis), Bael Fruit (Aegle marmelos), Bishop's weed (Trachyspermum ammi), )Bitter Chamomile (Matricaria chamomilla), Clove (Syzygium aromaticum), Ginger (Zingiber officinale), Holy Basil (Ocimum sanctum), Indian Acalypha (Acalypha indica), Datura (Datura innoxia), Mint (Mentha spp.), Betel leaves (Piper betle, Linn), Calendula (Calendula officinalis LINN), Chick weed (Trichobasis lychnidearum), Cucumber (cucumis sativus, Linn), Acasia arabica, Olive (Olea europea L.), Wild daisy, Cumin seeds (Cuminum cyminum), Curry leaves (Murraya koengi), Dill (Anethum graveolens), Indian mallow (Abutilon indicum), margosa (Azadirachta indica), Madhua (Madhuca indica), Tamarind (Tamarindus indicus) Turmeric (Curcuma longa), Winter cherry (Withania sommnifera), Zizyphus (Zizyphus jujuba), Pumpkin (Cucurbita pepo, Cucurbita maxima), Basswood ((Tilia americana)), Sweet flag (Acorus calamus), Amaranth (Amaranthus, spinosa), Arnica (Arnica Montana), American elder (Sambucus Canadensis), Betony (Stachys officinalis), Black berry (Eugenia jambolana), Calendula (Calendula officinalis LINN) Chamomile (Matricaria chamomilla), Club moss ((Lycopodium selago or clavatum), Dandelion (Taraxacum officinalem), Echinicea (Echinacea angustifolia), Eucalyptus (Eucalyptus globules), Golden seal (Hydrastis Canadensis), Fig wort, Comfrey (Symphytum officinale), Cowslip (Primula veris (L)), European sanicle (Sanicula europaea), European vervain (Verbena officinalis), Horse weed, Houseleek (Sempervivum tectorum, Linn), Larch (Larix laricina), Lungwort (Pulmonaria angustifolia), Onion (Alium cepa), Papaya (Carica papaya), Peach tree (Prunus persica, Pansy (Viola tricolor (LINN.), Pearly everlasting (Anaphalismargaritacea).

The carrier is a cream, gel, ointment, liquid, suspension, aerosol spray, gauze, fibrous wad, membrane, film, tape, plaster.

In accordance with another aspect of the invention, there is provided a method of making an antimicrobial formulation according to any one of the preceding claims, which includes the steps of
(1) making a carrier selected from a group of carriers which includes cream, gel, ointment, liquid, suspension, aerosol spray, gauze, fibrous wad, membrane, film, tape, plaster, cake in a conventional manner,
(2) making an aqueous dispersion of biologically stabilized silver nano particles in the size range of 1 to 100 nm;
(3) mixing a dispensed quantity of the said aqueous dispersion in the said carrier to form a homogenous matrix in which the concentration of the silver nano particles ranges between 1 to 6 ppm.

The aqueous dispersion of biologically stabilized silver nano particles is made by the steps of
(a) dissolving a salt of Silver in water having conductivity less than 3 micro siemens to obtain a solution in which the concentration of Silver ions is in the range of 20, 000 to 50 000 ppm,
(b) preparing a fresh filtered aqueous solution of biological tissue extract;
(c) diluting the aqueous solution with deionized water in the ratio ranging from 1:5 to 1:50 to form a solution having an open circuit potential between +0.2 and +0.2 volt and a pH between 5.5 to 7.5 and total organic carbon content at least 7,500 ppm;
(d) maintaining the said aqueous solution under continuous agitation at a temperature between 20 and 30 degrees Celsius;
(e) inoculating a minute quantity of the Silver salt solution in the said aqueous extract solution under continuous agitation such that the final concentration of the metal ion in the reaction mixture is in the range of 50 to 300 ppm;
(f) continuing the agitation for a period of 30 minutes to 3 hours in well illuminated conditions; to obtain a colloidal suspension of Silver nano particles;
(g) separating the nano particles from colloidal suspension by a known process such as centrifugation.

The process of making the biologically stabilized silver nano particles can be exemplified as follows

Using Labconco, USA water pro system with pre-filter, carbon filter and reverse osmosis membrane water was collected. The said water had the conductivity of 2.7 microSiemens as measured by the online digital meter fitted in the instrument.

50 whole flowers of Hibiscus rosasinensis Linn (48.37 gm wet wt) were macerated with 150 ml of deionized water in a blender (500 rpm) for 10 minutes to get a homogenous viscous suspension. This viscous suspension was filtered through Whatman No 1 filter paper under vacuum to obtain a clear 165 ml of viscous solution. From this an aliquot of 10 ml was diluted up to 100 ml using water.

An aliquot of 7 ml were removed and checked for open circuit potential at 25° C. on Electrochemical analyzer (CH Instruments 600B, USA) using a three-electrode system. Ag/AgCl _{(aq)} was used as reference electrode, Glassy carbon as working electrode (diameter 3 mm) and Pt wire (length 4 cm) as counter electrode. The value found was +0.15 Volt. Similarly pH of free flowing solution was checked using Digital pH meter (control Dynamics, India) and it was found to be 5.6.

The concentration of total organic carbon was measured using Beckman TOC analyzer and was found to be 22,180 ppm.

The synthesis of silver nanoparticle was carried out by Borohydride reduction method as described by Jin. R, Cao. Y. W., Kelly K. L., Schatz G. C., Zheng, J. G. and Chad A. Mirkin. (2001) Photoinduced conversion of silver nanospheres to nanoprisms. Science: 294; 1901-1903. Briefly, 10 ml of flower aqueous extract reacted with 100 μl of silver nitrate stock solution (100 mM) followed by addition of 100 μl of sodium borohydride (500 mM) which resulted in formation of a colloidal suspension.

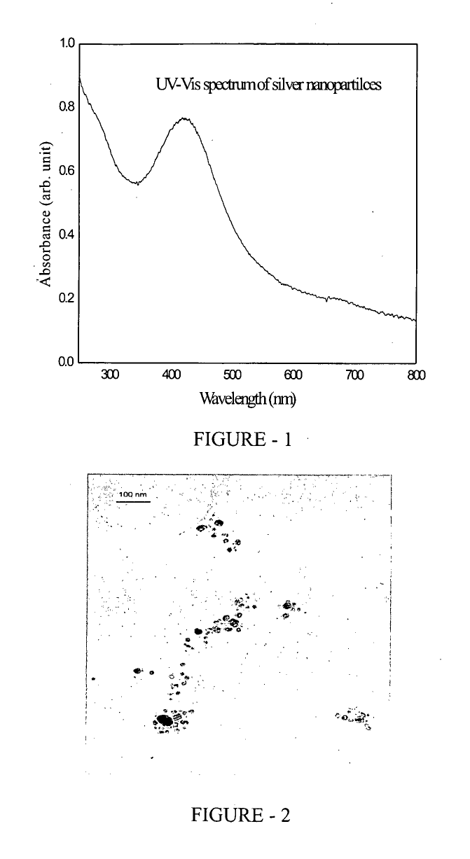

Sample of colloidal suspension was scanned from 200-800 nm using Diode Array spectrophotometer (Ocean Optics, USA). A peak at 410 nm was detected. This peak was characteristic plasmon peak for silver nano particle [FIG. 1 of the accompanying drawings], typically having average diameter of 5-120 nm.

Another aliquot of colloidal suspension was examined by Transmission electron microscopy (TEM) at 200 kV using Philips electron microscope equipped with field emission gun, i.e., CM200 FEG. TEM specimen was prepared by pipetting 2 μL of colloid solution onto a carbon coated copper grid and image was obtained. The average size seen in image was 10-20 nm. [FIG. 2 of the accompanying drawings]

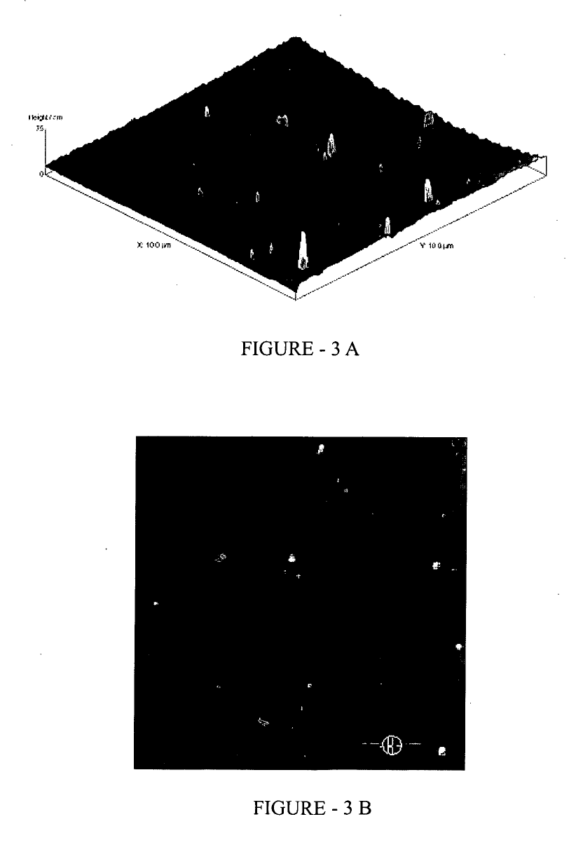

Atomic force microscopy (AFM) of the sample was performed using Nanonics MultiView 1000 AFM head with E scanner (Nanonics Imaging Ltd., Jerusalem, Israel). Sample was scanned in non-contact mode with a probe of 20 nm radius and a resonance frequency of 80 kHz. AFM images were captured, processed and analyzed with QUARTZ software, Version 1.00 (Cavendish Instruments Ltd., UK). For specimen, 5 μL of sample was placed on a 1-cm² glass slide (thickness 0.5 mm) and dried in laminar airflow before imaging. Uniform particles of 50-100 nm diameter and 125 nm height were observed as seen in FIG. 3a of the accompanying drawings which is the three dimension AFM view of a portion of the sample and 3b is a two dimensional view showing size analysis of a typical particle].

Evaluation of the Antimicrobial Activity

For the purposes of evaluation formulations were made as follows:

Liquid Suspension

The silver nano particle suspension produced as above was diluted with deionised water in separate containers in which the concentration of the biologically stabilized silver nano particles was measured to be in the range of 1.56 to 6 ppm.

Cream Based Ointment:

Liquid paraffin (400 ml), was mixed with zinc oxide (25 gm) and glycerine (25 gm) to obtain a homogenous mixture. Wax (50 gm), special wax (50 gm) and stearic acid (11 gm) were heated in a water bath set at 100° C. to form homogenous liquid mixture. The liquid paraffin mixture was poured into the wax mixture slowly, and stirred vigorously to get a homogenous mass. Lyophilised powder of biologically stabilized silver nano particles (5 mg) was introduced into the mass slowly and under continuous stirring to obtain a homogenous silver nano particles containing cream.

Gauze Piece

The biologically stabilized silver nano particles suspension or ointment produced as above were impregnated in sterilized gauze pieces.

The antimicrobial potential of biologically stabilized silver nano particles was evaluated on the basis of following procedures and tests.

Microorganisms. The following bacterial strains were used in the study: Escherichia coli ATCC 117, Pseudomonas aeruginosa ATCC 9027, Salmonella abony NCTC 6017, Salmonella typhimurium ATCC 23564, Klebsiella aerogenes ATCC 1950, Proteus vulgaris NCBI 4157, Staphylococcus aureus ATCC 6538P, Bacillus subtilis ATCC 6633. and Candida albicans [yeast].

Susceptibility testing. The minimum inhibitory concentration (MIC) of biologically stabilized silver nano particles for above-mentioned strains was performed according to the recommendations of the National Committee for Clinical Laboratory Standards (NCCLS) in ninety six well microtitre plates containing 200 μl MH broth. The concentration of silver in the wells ranged from 1.56-25 μg/ml. The log phase cell suspensions were diluted with saline and inoculated in the wells to give a final inoculum concentration of 1×10⁵ CFU/ml. The microtitre plates were incubated at 37° C. and were scored visually for growth/no growth after 24 h. The lowest concentration of silver inhibiting growth was recorded as the minimum inhibitory concentration (MIC). The medium from wells showing no visible growth was spot inoculated on MH agar plates and the plates were incubated for 24 h to determine the minimum silver concentration that is bactericidal (MBC).

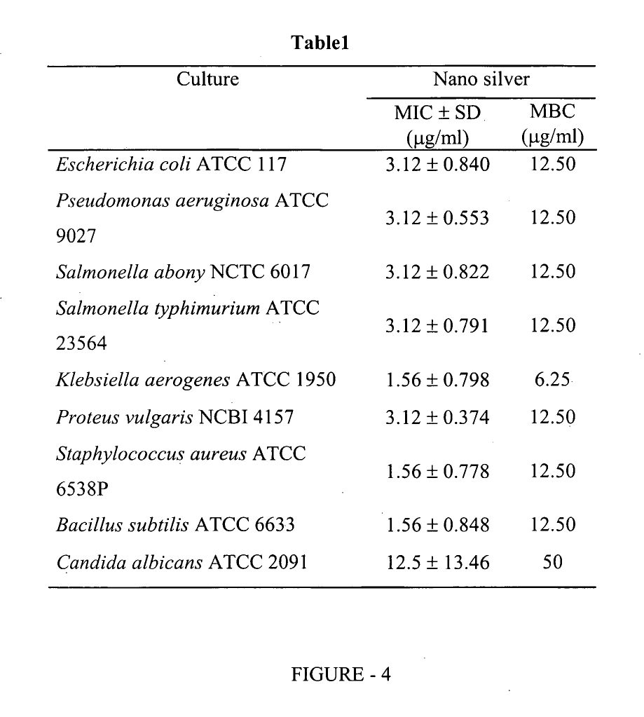

The results are seen in FIG. 4 [Table 1] of the accompanying drawings. The results show that MIC values for gram positive and gram negative bacteria range between 1.56 and 3.12 ppm of biologically stabilized silver nano particles, while MBC values range between 6.25 to 12.5 ppm of biologically stabilized silver nano particles. The MIC value for the yeast is 12.5 ppm and the MBC is 50 which shows that in the selected concentration there is no significant action on the yeast.

Effect of neutralizing agents on biologically stabilized silver nano particles. The neutralization of the activity of biologically stabilized silver nano particles in the presence of serum albumin, sodium chloride and sodium thioglycolate was tested in MHB. For this purpose one set of MHB was supplemented with three different concentrations of serum albumin (2%, 5%, 10%) along with 0.85% sodium chloride, in another set 0.1%, 0.5% and 1% sodium thioglycolate was added as suggested by Furr et al (1994). The MIC was determined as described above.

It was found that the MIC values remained unchanged in the presence of the above mentioned neutralizing agents.

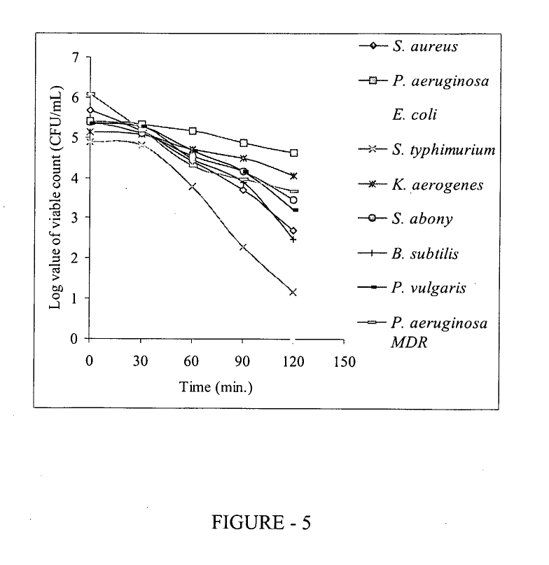

Time-kill kinetics. The bacterial cultures were inoculated (final cell density of 1×10⁵ CFU/ml) in 2 ml MH broth supplemented with appropriate amounts of biologically stabilized silver nano particles (at concentration corresponding to MBC for the respective cultures). After exposure to biologically stabilized silver nano particles at specified time intervals (ca. 0, 30, 60, 90 and 120 min) 0.1 ml samples were removed, serially diluted, and plated on MH agar plates. The total viable count (TVC) was determined after incubating the plates at 37° C. for 24 h. All experiments were performed in four replicates. Kill curves were constructed by plotting the log₁₀ of CFU/ml versus time. These kill curves are shown in FIG. 5 of the accompanying drawings.

For all the bacterial cultures tested, the total viable cell population reduced by 90% within a short exposure time of 2 h. The data suggests that biologically stabilized silver nano particles effectively inhibit the growth of Gram-negative and Grm-positive bacteria including multi drug resistant strain of Pseudomonas aeruginosa.

Post biologically stabilized silver nano particles effect. Post silver nano particles effect was studied using a spectrophotometric method. Briefly, all the bacterial strains (10⁵ CFU/ml) were exposed to 4× MBC of biologically stabilized silver nano particles for 1 h at 37° C. Cultures not exposed to biologically stabilized silver nano particles served as controls in the experiment The suspensions were centrifuged at 3000×g for 10 min. and the pellets were washed several times with physiological saline to remove any traces of silver nano particles. Colony counts were taken at time zero (N_{inic}) and after removal of biologically stabilized silver nanoparticles (Nₙₐₙₒₛᵢₗᵥₑᵣ). The culture pellets were then suspended in MHB and growth of the culture was monitored periodically by O.D measurements at 660 nm. All the cultures were incubated at 37° C. with agitation and the O.D was measured after every 1 h. Post biologically stabilized silver nano particles effect was calculated as the difference in the time required for attaining one log scale increase in the CFU of biologically stabilized silver nano particles—exposed and unexposed test culture.

Calculation of the Post Biologically Stabilized Silver Nano Particles Effect

Once N_{inic} and Nₙₐₙₒₛᵢₗᵥₑᵣ were determined and the growth of the control and exposed cultures were spectrophotometrically monitored, following steps were carried out. (i) Plotting, on semi-logarithmic paper, of the spectrophotometric growth curves of the control and post-exposure cultures, representing optical density (O.D) along the y-axis and time along the x-axis customarily the first meaningful reading of O.D can be taken for the control culture at 4 or 5 h after the initial time (t_{inic}=0). (ii) Determination of the generation time (t_{g}) from spectrophotometric growth curves. (iii) The calculation of bactericidal effect (r):
r=N_{inic}/Nₙₐₙₒₛᵢₗᵥₑᵣ

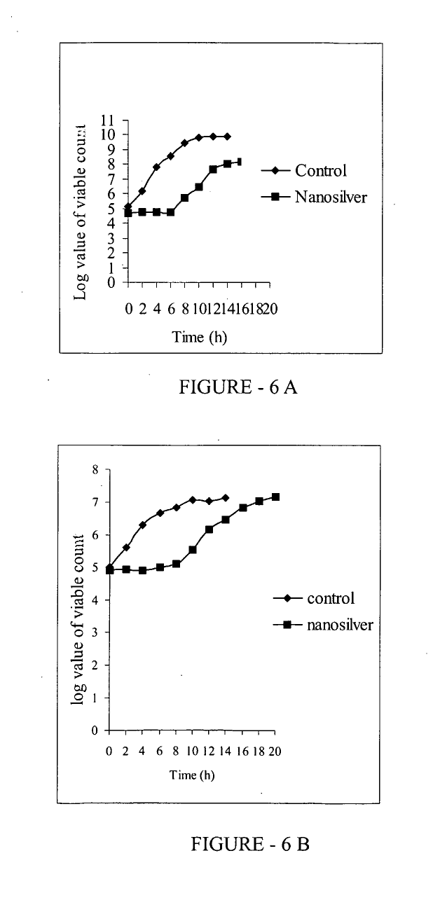

(iv) Graphical determination of the time separation of the spectrophotometric growth curves of the control culture and the post-exposure culture (tₛₑₚ). (v) Calculation of the post biologically stabilized silver nano particles effect was done according to the following formula:
Post biologically stabilized silver nano particles effect=tsep−tₑₓₚₒ−t_{g} log r/log 2

where tₛₑₚ is the separation time of the spectrophotometric growth curves of the control culture and the post-exposure culture; tₑₓₚₒ is the exposure time equivalent to 1 h duration and t_{recrt} is the theoretical time that the treated culture takes for its viability count (Nₐₙₜᵢ) to match the initial count (N_{inic}), r is bactericidal effect and t_{g} is generation time. FIGS. 6a and 6b of the accompanying drawings show the post biologically stabilized silver nano particles effect on a gram negative bacterial culture and a yeast respectively. Post biologically stabilized silver nano particles effect was found to be 6-8 h as indicated by lag phase in the growth curves.

Interaction of drugs with biologically stabilized silver nano particles. A two-dimensional checkerboard macrodilution technique was used to characterize interactions between biologically stabilized silver nano particles and drug (viz. Gentamicin, Penicillin, Cefotaxime, Ceptazidime, Kanamycin, Vancomycin). six experiments were performed for Pseudomonas aeruginosa MDR strain described above. Inoculum was prepared similarly to those for susceptibility testing. Individual drug with biologically stabilized silver nano particles were diluted in serial twofold dilutions, and concentrations ranged from four fold below to four folds above the MIC. The highest dilution of the drug combination with biologically stabilized silver nano particles that inhibits the visible growth of the test organism was regarded as the fractional inhibitory concentration (FIC). The fractional inhibitory concentration (FIC) index (FICI) was used to define the interaction between the two drugs. The FICI is the sum of the FICs of each of the drugs. The FIC was calculated as follows: MIC of the drug tested in combination/MIC of the drug tested alone. The interaction was defined as synergistic if the FICI was 0.5, as additive if the FICI was >0.5 to 1.0, as indifferent if the FICI was >1.0 to 2.0, and as antagonistic if the FICI was >2.0.

The mean FICI values obtained during the course of this work indicated that the action of biologically stabilized silver nano particles was synergistic with cefotaxime and cephalosporin, partially synergistic with ceptazidime and indifferent to gentamicin, penicillin and ampicillin. However, an antagonistic effect was seen in combination with kanamycin and vancomycin.

Antimicrobial activity of biologically stabilized silver nano particles on Gauze piece. Gauze pieces (2 cm×2 cm) were autoclaved and wetted with 100 μl of biologically stabilized silver nano particles suspension or by applying an ointment containing biologically stabilized silver nano particles (both having silver nano particles concentration of 4× MIC for respective cultures) and inoculated with 10⁵ cells. Controls with sterile physiological saline were run simultaneously. The gauze pieces were put in sterile petri dishes and kept in a humidified incubator set at 37° C. Viability of the cultures was checked at 0 h and at an interval of 4 h for 24 h. For this, a gauze piece was removed, placed in 10 ml saline, vortexed and the suspension was serially diluted and plated on nutrient agar plate. The TVC was determined after 24 h incubation at 37° C. Six parallel experiments were carried out at each reaction time for both treated and control gauze piece.

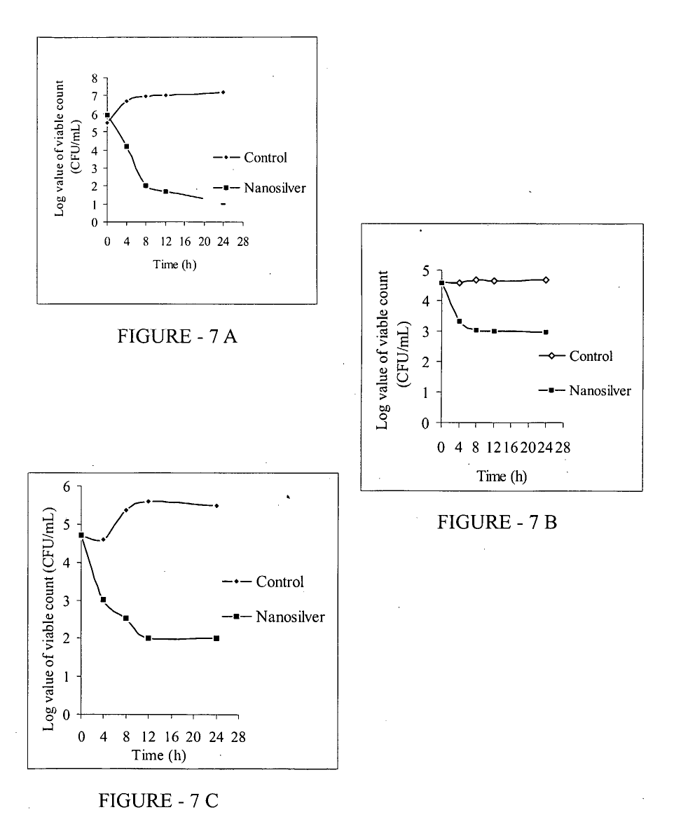

The log₁₀ number of organisms recovered from each type of gauze piece was calculated for all six parallel experiments (one colony was assumed when zero colonies were detected). The results are depicted as mean Log value versus reaction time in FIGS. 7A, 7B and 7C of the accompanied drawings.

The results obtained showed that the time required for >97% reduction in the population of test organisms was 8 h.

In vitro cytotoxicity testing., Human leukemic cell line K562, hepatocellular carcinoma cell line HEPG2 and nouse fibroblasts L929 were routinely cultured in Dulbecco's modified Eagle medium (DMEM, Sigma, USA) supplemented with 10% fetal calf serum and 1% of commercial preparation of antibiotic-antimycotic (PenStrep, Sigma, USA). Cultures were maintained at 37° C. in a 5% CO₂ atmosphere.

The comparative effect of four preparations namely electro chemically synthesized silver nano particles stabilized with glycerol (EC-Gly), electro chemically synthesized silver nano particles stabilized with poly vinyl pyrrolidone (EC-PVP), biologically stabilized silver nano particles (Chem-Bio) and silver nitrate (AgNO₃), were checked on the cell proliferation and viability on the above three cell lines using XTT assay kit (Roche Molecular Biochemicals, Germany). Briefly, Microtiter plates (96 wells) containing DMEM were seeded at an initial cell density of 1×10⁵ cell/ml. The cells were allowed to proliferate for 24 h at 37° C, 5% CO₂. After incubation, the supernatant medium was carefully removed and sterile DMEM supplemented with nano silver in the range 1.5-15 μg/ml was added. Plates were further incubated for 48 h at 37° C. in 5% CO₂ atmosphere. After incubation 50 μl of XTT reagent was added to each well and plates were incubated at 37° C., 5% CO₂ atmosphere for 4 h. The color developed due to formation of formazan was quantified using ELISA reader (μQuant, Biotech Instruments) at 450 nm. The plates were scanned at 690 nm so as to provide correction for absorbance by cells. XTT assay was performed in 6 replicates with appropriate controls without silver nano particles.

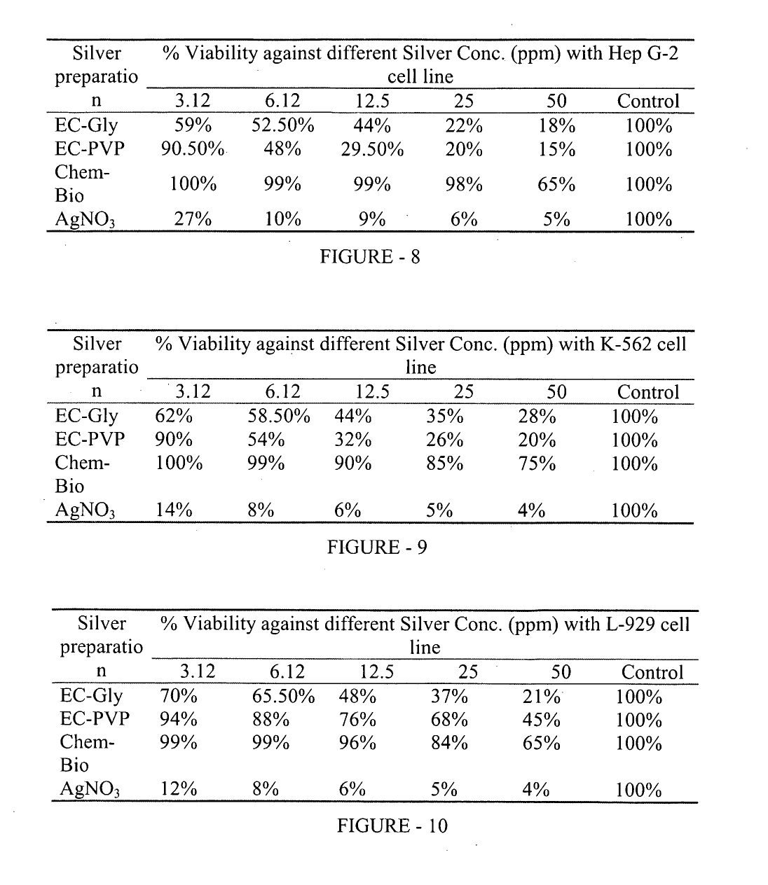

The results obtained are shown in the tables as seen in FIGS. 8, 9 and 10 of the accompanying drawings.

It is clearly seen that biologically stabilized silver nano particles are non toxic to all the three cell lines in the concentration of 1 to 6 ppm. It can be also be seen that chemically stabilized silver nano particles as well as silver nitrate are highly cytotoxic even at a concentration of 3.12 ppm.

Thus in the experimentation done in accordance with this invention, biologically stabilized silver nano particles are found to have a broad spectrum antimicrobial effect in the concentration range of 1-6 ppm. Further, in this range of concentrations the biologically stabilized silver nano particles do not show in vitro cytotoxicity.

The biologically stabilized silver nano particles described here could have applications in the treatment of burn wounds, as coating material for various medical devices such as catheters, heart valves, bioactive glasses coated sutures and orthopedic devices. Its non-medical applications could be in water and air purification systems.

The biologically stabilized silver nano particles in accordance with this invention can be employed as a germicide and antibiotic for various bacterial infections.

Thus the a formulation made with the biologically stabilized silver nano particles may be useful in the treatment of Anthrax, Athlete's Foot, Boils, Candida, Cerebro-spinal meningitis, Colitis, Cystitis, Dermatitis, Diphtheria, Diplococcus, E. Coli, Gonorrhea, Impetigo, Infection, Pneumococci, Ringworm, Shingles, Staphylococci, Tuberculosis, Warts, Whooping Cough.

The biologically stabilized silver nano particles may be made into suspension/solution wherein the solvent may be purified water, water for injection as applicable to the sterile preparation and any other non-aqueous co-solvents may be such as polyglycols, alcohols, inert liquefied gases and other halogen carbon related compounds as like.

The other excipients may include surfactants, suspending agents, and viscosity modifying agents, waxes, cellulosic polymers, carbopols and optionally preservatives, buffering agents, osmotic adjusting agents or tonicifying adjusting agents, hydrocarbons and low boiling point solvents.

The excipients in the formulations may includes polysorbate, carbopols, hydroxypropyl methyl cellulose, petrolactum base, waxes, sodium chloride, mannitol, citric acids, phosphates, acetates, benzylalocohols, butyratehydroxytoluene (BHT), butyrated hydroxyanisone (BHA), glycols such as glycerin, polyethylene glycols, propylene glycols sorbitol, inert gases such as nitrogen, hydrogen and others, hydrocarbons may be ethanol, butanols, and others, flurocarbons.

The formulations may comprise of eye drops, eardrops, nose drops, solutions, ointments, creams, lotions and other preparation for dressings of burn or infections.

The said formulations those meant for external application to the infected area may also contain other synergistic active ingredients such as rubifacients that may be at least one selected from menthol, methyl salicylate, oleum lini, capsaicin.

This solution along with the low boiling solvents or compressed liquefied gases or hydrocarbons or combinations of any two may be placed in a pressurized system by using suitable machine wherein, the drug is sprayed into the infected area for instant action.

The ointment can be prepared by using the solution/suspension of the biologically stabilized silver nano particles with the said active excipients and may be along with the aforesaid co solvents. The viscosity modifying agents are used in suitable concentration so as to obtain the desire viscosity as per the requirement of the formulation.

The external formulation may also be prepared by using natural ingredients without preservatives. The fine sized particles in the formulation provide better bioavailability and absorption.

1. A non-cytotoxic antimicrobial formulation comprising
a) biologically stabilized silver nano particles in the size range of 1 to 100 nm; and
b) a carrier in which the concentration of the said biologically stabilized silver nano particles is 1 to 6 ppm.

2. An antimicrobial formulation of claim 1, wherein the silver nano particles are biologically stabilized with an aqueous solution of macerated plant tissue cells.

3. An antimicrobial formulation of claim 2 wherein the aqueous solution is diluted with deionised water to ten fold.

4. An antimicrobial formulation of claim 2, wherein the plant tissue is at least one plant tissue selected from the group consisting of leaves, roots, stems, flowers and fruits of the following plants Alfa alfa, Babul (Acacia arabica), Coriander (Coriandrum sativum), East Indian Rosebay (Ervatamia coronaria), Hog weed (Boerhavia diffusa), Indian Barberry (Berberris aristata), Marigold (Calendula officinals), Parsley (Petroselinum sativum), Rough Chaff (Achyranthes aspera), Tenner's Cassia (Cassia auriculata), Lavender, Bahera (Terminalia belerica), Fennel (Fenniculum vulgare), Horsetail (Equisetum arvense), Raspberry (Rubus idaeus), Aloe vera (Aloe barbadenesis), Golden seal (Hydrastis canadensis), Garlic (Allium sativum), Echinacea spp., Eyebright (Euphrasis officinalis), Bael Fruit (Aegle marmelos), Bishop's weed (Trachyspermum ammi), Bitter Chamomile (Matricaria chamomilla), Clove (Syzygium aromaticum), Ginger (Zingiber officinale), Holy Basil (Ocimum sanctum), Indian Acalypha (Acalypha indica), Datura (Datura innoxia), Mint (Mentha spp.), Betel leaves (Piper betle, Linn), Calendula (Calendula officinalis LINN), Chick weed (Trichobasis lychnidearum), Cucumber (cucumis sativus, Linn), Acasia arabica, Olive (Olea europea L.), Wild daisy, Cumin seeds (Cuminum cyminum), Curry leaves (Murrava koengi), Dill (Anethum graveolens), Indian mallow (Abutilon indicum), margosa (Azadidirachta indica), Madhua (Madhuca indica), Tamarind (tamarindus indicus) Turmeric (Curcuma longa), Winter cherry (Withania sommnifera), Zizyphus (Zizyphus jujuba), Pumpkin (Cucurbita pepo, Cucurbita maxima), Basswood ((Tilia americana)), Sweet flag (Acorus calamus), Amaranth (Amaranthus, spinosa), Arnica (Arnica Montana), American elder (Sambucus Canadensis), Betony (Stachys officinalis), Black berry (Eugenia jambolana), Calendula (Calendula officinalis LINN) Chamomile (Matricaria chamomilla), Club moss (Lycopodium selago or clavatum), Dandelion (Taraxacum officinalem), Echinacea (Echinacea angustifolia), Eucalyptus (Eucalyptus globules), Golden seal (Hydrastis Canadensis), Fig wort, Comfrey (Symphytum officinale), Cowslip (Primula veris (L)), European sanicle (Sanicula europaea), European vervain (Verbena officinalis), Horse weed, Houseleek (Sempervivum tectorum, Linn), Larch (Larix laricina), Lungwort (Pulmonaria angustifolia), Onion (Alium cepa), Papaya (Carica papaya), Peach tree (Prunus persica) Pansy (Viola tricolor (LINN.) and Pearly everlasting (Anaphalismargaritacea).

5. An antimicrobial formulation of claim 1, in which the carrier is selected from the group consisting of cream, gel, ointment, liquid, suspension, aerosol spray, gauze, fibrous wad, membrane, film, tape and plaster.

6. A method of making an antimicrobial formulation of claim 1 comprising
a) selecting a carrier selected from the group consisting of cream, gel, ointment, liquid, suspension, aerosol spray, gauze, fibrous wad, membrane, film, tape and cake in a conventional manner,
b) making an aqueous dispersion of biologically stabilized silver nano particles of 1 to 100 nm; and
c) mixing a dispensed quantity of the said aqueous dispersion in the said carrier to form a homogenous matrix in which the concentration of the biologically stabilized silver nano particles is between 1 to 6 ppm.

7. A method of making an antimicrobial formulation of claim 6, wherein the aqueous dispersion of biologically stabilized silver nano particles comprises
a) dissolving a salt of silver in water having conductivity less than 3 micro siemens to obtain a solution in which the concentration of silver ions is in the range of 20,000 to 50,000 ppm,
b) preparing a fresh filtered aqueous solution of biological tissue extract;
c) diluting the aqueous solution with deionized water in the ratio ranging from 1:5 to 1:50 to form a solution having an open circuit potential between +0.2 and +0.2 volt and a pH between 5.5 to 7.5 and total organic carbon content at least 7,500 ppm;
d) maintaining the said aqueous solution under continuous agitation at a temperature of 20 the 30° C.;
e) inoculating a minute quantity of the silver salt solution in the said aqueous extract solution under continuous agitation so that the final concentration of the metal ion in the reaction mixture is 50 to 300 ppm;
f) continuing the agitation for 30 minutes to 3 hours in well illuminated conditions to obtain a colloidal suspension of silver nano particles; and
g) separating the nano particles from colloidal suspension.
